SEQUENCE LISTING (12 sequences)

SEQ ID NO: 1 - DNA, 21 bp, Artificial Sequence
  This is a chemically synthesized mixed DNA/RNA sequence, the backbone is ribose-based except for deoxythymidines at positions 20 and 21
cuuacgcugaguacuucgatt

SEQ ID NO: 2 - DNA, 21 bp, Artificial Sequence
  This is a chemically synthesized mixed DNA/RNA sequence, the backbone is ribose-based except for deoxythymidines at positions 20 and 21
ucgaaguacucagcguaagtt

SEQ ID NO: 3 - RNA, 21 nt, Artificial Sequence
  Chemically synthesized small interfering RNA
cgagaaccagucuuaugacuu

SEQ ID NO: 4 - RNA, 21 nt, Artificial Sequence
  Chemically synthesized small interfering RNA
gucauaagaaggguucugcuu

SEQ ID NO: 5 - RNA, 21 nt, Artificial Sequence
  Chemically synthesized small interfering RNA
gcagaaccagucuuaugacuu

SEQ ID NO: 6 - RNA, 21 nt, Artificial Sequence
  Chemically synthesized small interfering RNA
gucauaagacugguugugcuu

SEQ ID NO: 7 - RNA, 21 nt, Artificial Sequence
  Chemically synthesized small interfering RNA
uagcgacuaaacacaucaauu

SEQ ID NO: 8 - RNA, 21 nt, Artificial Sequence
  Chemically synthesized small interfering RNA
uugauguguuuagucgcuauu

SEQ ID NO: 9 - DNA, 23 bp, Artificial Sequence
  PCR primer
cgactagttatgatcagagcagt

SEQ ID NO: 10 - DNA, 23 bp, Artificial Sequence
  PCR primer
ccgttgctctgtattcttactga

SEQ ID NO: 11 - DNA, 18 bp, Artificial Sequence
  PCR primer
gcaccccgtgctgctgac

SEQ ID NO: 12 - DNA, 18 bp, Artificial Sequence
  PCR primer
cagtggtacggccagagg

(19) United States
(12) Patent Application Publication
(10) Pub. No.: US 2007/0202076 A1
(43) Pub. Date: Aug. 30, 2007

(54) Methods and compositions for the targeted delivery of therapeutics

(76) Inventors: Timothy J. Triche, Los Angeles, CA (US); Siwen Hu, Pasadena, CA (US)

Correspondence Address: OLSON & HIERL, LTD., 20 NORTH WACKER DRIVE, 36TH FLOOR, CHICAGO, IL, 60606, US

(21) Appl. No.: 11/388,400

(22) Filed: Mar. 24, 2006

Related U.S. Application Data
(63) Continuation of application No. 11/234,827, filed on Sep. 23, 2005, now abandoned.
(60) Provisional application No. 60/612,326, filed on Sep. 23, 2004.

(51) Int. Cl. A61K 31/765; A61K 31/715

(52) U.S. Cl. 424/78.37; 514/54

(57) ABSTRACT

Disclosed herein are compositions and methods for the targeted delivery of a therapeutic agent. In one aspect, the invention pertains to glycopolymer-based particles complexed with a nucleic acid-based therapeutic. Other aspects of the invention relate to methods for treating various conditions by administering the particle compositions of the invention. In some embodiments, a cyclodextrin-based particle is used to deliver siRNA against one or more oncogenes.

### RELATED APPLICATIONS

This application claims the benefit of the U.S. Provisional Application entitled, “USING A CYCLODEXTRIN-CONTAINING POLYCATION TO DELIVER siRNA TARGETING THE BREAKPOINT OF EWS-FLI1 FOR TREATING PATIENTS WITH EWING'S FAMILY TUMORS,” filed Sep. 23, 2004, attorney docket No. CIT-4210-P, which is hereby incorporated by reference in its entirety.

### BACKGROUND OF THE INVENTION

1. Field of the Invention

The present invention relates generally to non-viral methods and compositions for the targeted delivery of therapeutic agents.

2. Description of the Related Art

The delivery of therapeutic agents in vivo is often complicated by limitations with regard to solubility, stability, toxicity, and other factors. A wide variety of drug delivery systems have been developed to overcome these obstacles, but each typically suffers from disadvantages, such as low stability, poor tissue specificity, toxicity, and reproducibility. Thus, there is a need for drug delivery systems that allow for the safe, biocompatible, stable and efficient delivery of a wide variety of therapeutic agents.

### BRIEF DESCRIPTION OF THE DRAWINGS

FIG. 1. Schematic illustration of the delivery system. (a) Components of the delivery system. The cyclodextrin-containing polycation (CDP) condenses siRNA and protects it from nuclease degradation. The adamantane-poly (ethylene glycol) (AD-PEG) conjugate stabilizes the particles in physiological fluids via inclusion compound formation. The AD-PEG-transferrin (AD-PEG-Tf) conjugate confers a targeting ligand to particles, promoting their uptake by cells overexpressing the cell-surface transferrin receptor (TfR). (b) Assembly of the non-targeted and targeted particles. For non-targeted particles, CDP and AD-PEG are combined and added to siRNA to generate stable but non-targeted polyplexes. For targeted particles, CDP, AD-PEG, and AD-PEG-Tf are combined and added to siRNA to generate stable, targeted particles.

FIG. 2. In vitro down-regulation of EWS-FLI1 in cultured TC71 EFT cells. (a) Quantification of Western blot analysis. Cultured TC71 cells were exposed to siEFBP2-containing formulations made with Oligofectamine (OFA) or cyclodextrin-containing polycation (CDP) for 4 h. At 48 h post-transfection, cells were lysed and total cell protein was denatured, electrophoresed, and transferred to a PVDF membrane that was probed with antibodies to EWS-FLI1 or actin (siEFBP2mut: mutant negative control). Average band intensities were determined by densitometry and the ratio of EWS-FLI1 to actin intensities was calculated. (b) Determination of the relative surface TfR level in TC71 cells. Cultured TC71 cells were incubated in medium containing fluorescein-labeled transferrin (Tf-FITC); uptake was assessed by flow cytometry. This experiment was also performed on cell lines known to express high and low levels of TfR (HeLa and A2780, respectively) for comparison.

FIG. 3. Establishment of a metastatic EFT model in mice. (a) NOD/scid mice injected with TC71-LUC cells developed metastatic tumors. Mice were injected with TC71-LUC cells via the tail vein. At various time points after injection, mice were anesthetized, injected with D-Luciferin and imaged using a Xenogen IVIS 100 bioluminescence imaging system. (b) MRI confirmation of EFT engraftments. Tumor-bearing mice were anesthetized, injected with contrast agent and imaged. Tumor locations observed by MRI corresponded to bioluminescent signal.

FIG. 4. Effect of siEFBP2 formulations on growth of metastasized EFT in mice. (a) Reduced bioluminescence in mice receiving formulated siRNA targeting EWS-FLI1 (siEFBP2). siEFBP2 was formulated and targeted as described in FIG. 1 and administered by low-pressure tail vein (LPTV) injection on three consecutive days (Day 35, 36, and 37, red arrows) after injection of TC71-LUC cells. Transient reduction in bioluminescence was observed on days 36 and 37. (b) EWS-FLI1 RNA level in tumors after two consecutive injections of fully formulated siRNA. Formulated siEFBP2 or siCON1 were administered by LPTV injection on two consecutive days (Days 19 and 20) after injection of TC71-LUC cells. Tumors were harvested on the third day. RNA were extracted and EWS-FLI1 level was determined by Q-RT-PCR.

FIG. 5. Effect of long-term delivery of siRNA formulations on growth of metastasized EFT in mice. (a) Bioluminescence imaging of NOD/scid mice treated twice-weekly with formulated siRNA for four weeks. Starting immediately after injection of TC71-LUC cells, mice were treated with formulations containing siRNA targeting EWS-FLI1 (siEFBP2) or a non-targeting control sequence (siCON1) twice-weekly for four weeks. The bioluminescence of these mice was monitored twice-weekly. All images shown are for 3.5 weeks after beginning of treatment and have identical scales for image comparison. (b) Growth curves for engrafted tumors. The median integrated tumor bioluminescent signal (photons/sec) for each treatment group [n=8-10] is plotted versus time after cell injection (d). [Treatment groups: A, 5% (w/v) glucose only (D5W); B, naked siEFBP2; C, targeted, formulated siCON1; D, targeted, formulated siEFBP2; E, non-targeted, formulated siEFBP2.]

FIG. 6. Formulated siRNA failed to exhibit toxicity or elicit an immune response in mice. (a) and (b)—CBC and liver panel results for C57BL/6 mice receiving formulations showed no toxicity or immune response. Female C57BL/6 mice received a single administration of formulated siRNA. At 2 h or 24 h post-treatment, blood was drawn by cardiac puncture and plasma was isolated. Whole blood was used for determination of platelet (PLT) and white blood cell (WBC) counts. Plasma was used for measurement of aspartate aminotransferase (AST), alanine aminotransferase (ALT), alkaline phosphatase (ALKP), creatinine (CRE), and blood urea nitrogen (BUN). The averages of triplicate mice for each time point are plotted; error bars represent standard deviations. FIG. 6(a) shows results for aspartate aminotransferase (AST), alanine aminotransferase (ALT), alkaline phosphatase (ALK), and platelets (PLT). FIG. 6(b) shows results for white blood cells (WBC), blood urea nitrogen (BUN), and creatinine (CRE). (c) and (d)—Cytokine ELISA results for C57BL/6 mice receiving formulations showed no up-regulation of IL-12 (FIG. 6 (c)) or IFN-α (FIG. 6(d)). The plasma levels of interleukin-12 (IL-12 (p40)) and interferon-alpha (IFN-α) in mice described above were measured by ELISA. [Treatment groups: A, 5% (w/v) glucose only (D5W); B, naked siEFBP2; C, targeted, formulated siCON1; D, targeted, formulated siEFBP2; E, non-targeted, formulated siEFBP2; Wild-type, uninjected; 2, blood drawn 2 h after injection; 24, blood drawn 24 h after injection.] (e) H&E staining of major organs of the NOD/scid mice after long-term treatment Major organs were collected, formalin-fixed and processed for routine hematoxylin and eosin staining using standard methods. Images were collected using a Nikon epifluorescent microscope with a DP11 digital camera.

### DESCRIPTION OF THE INVENTION

Disclosed herein are methods and compositions for delivering therapeutics for the treatment of various conditions. In one aspect, the application discloses particles comprising biocompatible polymers, and uses of such particles to deliver small molecule drugs, nucleic acids, and other therapeutics. In some preferred embodiments, the particles are comprised of a backbone glycopolymer, such as a cyclodextrin polymer, and a polymeric cross-linker that links two or more of the backbone polymers. In some embodiments, the polymer-based particles are biodegradable under the conditions of their intended use. In certain embodiments, the particles have an average diameter of less than about 100 nanometers, more preferably less than about 50 nanometers, and greater than about 10 nanometers. Without being limited by any particular theory, it is believed that the hydrophilic surface of cyclodextrin-based particles provides water solubility while the hydrophobic cavity provides a stable environment in which to enclose, envelope or entrap one or more therapeutic agents.

As used herein, “therapeutic agent” includes any synthetic or naturally occurring compound or composition of matter which produces a desired response when administered to an organism (human or animal). In some embodiments, a desired response is the alleviation and/or prophylaxis of one or more symptoms and/or indicators of a disease or condition targeted for treatment. Useful therapeutic agents can comprise small molecule drugs, vaccines, biopharmaceuticals, including proteins, peptides, lipids, carbohydrates, hormones, nucleic acids, and the like, and/or any molecule capable of producing a desired therapeutic effect. The invention is not limited as to the nature of the therapeutic agent. In some embodiments, the therapeutic agent has a substantially lower solubility and/or stability under physiologically relevant conditions (e.g., conditions typical of the targeted cell type, tissue, organ, etc.) than the solubility/stability of the agent when associated with a particle of the invention.

In some embodiments, glycopolymer-based particles are surface modified with one or more moieties that confer one or more advantageous properties to the particles, including but not limited to increased solubility, enhanced stability, enhanced therapeutic index, reduced toxicity, and/or reductions in the degree or nature of side effects. In some embodiments, the particles are surface modified with one or more ligands against a molecular target. Examples of suitable ligands include ligands of cell-surface receptors, molecules that bind cell-surface glycoproteins, and antibodies or antibody fragments against cell surface molecules. In some embodiments, the particles are imported into target cells, for example by endocytosis. In some preferred embodiments, particles used in therapeutic methods, as well as methods used to prepare and administer such particles, are described in U.S. Pat. Nos. 6,884,789 and 6,509,323; and in U.S. Patent Publication Nos. 20050136430; 20040109888; 20040063654 and 20030157030, each of which is hereby incorporated by reference in their entirety.

In some embodiments, the particles are capable of administration orally, intravenously, via inhalation (e.g., pulmonary or nasal administration), and/or by other routes of administration. In some preferred embodiments, particle compositions are stable under physiological conditions, such as physiological salt, temperature, and pH conditions, for a duration suitable for treating the condition targeted for treatment. In some preferred embodiments, the half-life and/or solubility of a particle of the invention is substantially greater than the half-life and/or solubility of the agent delivered by the particle under the same conditions.

In some preferred embodiments, particles allow for repeated administration without causing a substantial immune response. For example, in some preferred embodiments, administration of the compositions of the invention results in no detectable interferon response, as is typical with known lipid-based delivery methods.

In some preferred embodiments, therapeutic agents delivered by methods described herein have a limited half-life of effectiveness in vivo (e.g., less than the desired dosing interval), such that the therapeutic effect and extent of treatment is substantially determined by the dosage and/or frequency of administration. For example, in some embodiments, methods allow for treatment with a rapid onset of action and a rapid termination of treatment after the therapeutic goal has been reached (e.g., after the regression of a tumor). Advantageously, the methods and compositions allow for the calibration of treatment so as to provide the minimum therapeutically effective amount of a therapeutic agent.

In some preferred embodiments, methods are provided for treating cancer comprising administering a particle caring an RNAi-based therapeutic which sequence specifically down-regulates, inhibits or abolishes expression of one or more genes. In some embodiments, the RNAi therapeutic is a double-stranded short interfering RNA (siRNA) comprising about 10 to about 40 base pairs, and more preferably from about 15 to about 28 base pairs. In various embodiments, the gene targeted by the RNAi-based therapeutic is selected from the group including, but not limited to, cyclin dependent kinases, c-myb, c-myc, GSK3-beta, proliferating cell nuclear antigen (PCNA), transforming growth factor-beta (TGF-beta), nuclear factor kappaB (NF-B), E2F, HER-2/neu, PKA, TGF-alpha, EGFR, TGF-beta, IGFIR, P12, MDM2, BRCA, Bcl-2 and other Bcl family members, VEGF, MDR, ferritin, transferrin receptor, IRE, C-fos, HSP27 and other HSP family members, C-raf, and metallothionein genes.

In some preferred embodiments, the gene targeted by the RNAi-based therapeutic is an oncogene that is tumor-specific and/or the product of a translocation. In some preferred embodiments, the oncogene is specific for a Ewing's Family Tumor, such as the genes listed in Hu-Lieskovan et al., Cancer Res., 65(11): 4633-44 (2005), which is herein incorporated by reference in its entirety. In some preferred embodiments, the oncogene is selected from the group including, but not limited to, WNT-5a, 2CITED, C-Myc, Id2, MSX1, Cyclin D1, CEBPβ, PTPR1A, PTPNS1, and PKCβ1.

In some embodiments, methods provide targeted delivery of siRNA therapeutics to tumor cells, hepatocytes, and/or other cell types via systemic administration. Advantageously, methods for targeting RNAi-based therapeutics provide enhanced safety, potency, specificity, and/or other desirable attributes relative to known methods.

In some embodiments, the RNAi-based therapeutic, and methods for preparing and administering such therapeutics, are described in U.S. Patent Publication Nos. 20050136430; 20040063654; and 20030157030, each of which is hereby incorporated by reference in their entirety.

Some aspects of the present invention are exemplified below.

The development of effective, systemic therapies for metastatic cancer is highly desired. We show here that the systemic delivery of sequence-specific small interfering RNA (siRNA) against the EWS-FLI1 gene product by a targeted, non-viral delivery system dramatically inhibits tumor growth in a murine model of metastatic Ewing's sarcoma. The non-viral delivery system utilizes a cyclodextrin-containing polycation to bind and protect siRNA and transferrin as a targeting ligand for delivery to transferrin-receptor-expressing tumor cells. Removal of the targeting ligand or the use of a control siRNA sequence eliminates the anti-tumor effects. Additionally, no abnormalities in interleukin-12 and interferon-alpha, liver and kidney function tests, complete blood counts, or pathology of major organs are observed from long-term, low-pressure, low-volume tail-vein administrations. These data provide strong evidence for the safety and efficacy of this targeted, non-viral siRNA delivery system.

Treatment-resistant metastases are the ultimate cause of death in most cancer patients. Ewing's family of tumors (EFT), a poorly differentiated mesenchymal malignancy that arises in bone or soft tissue, is a particularly cogent example. Historical data show that virtually all patients die from metastases (e.g., <5% survival after localized therapy(1)). Systemic chemotherapy has markedly improved survival of patients with localized disease, but patients with metastatic disease rarely benefit (2). A major factor contributing to this outcome is the development of multi-drug resistance by the time patients are treated for metastasis.

Specific chromosomal translocations are associated with numerous hematopoietic and solid tumors. The translocation t(11;22) is commonly detected in EFT and produces the chimeric EWS-FLI1 fusion gene found in 85% of EFT patients(2). Functionally equivalent chimeric genes are found in virtually all EFTs(3). EWS-FLI1 is thought to be a transcriptional activator and plays a significant role in tumorigenesis of EFT(4, 5). Reduction of the EWS-FLI1 protein in EFT cells in vitro or in subcutaneous xenograft tumors by antisense oligonucleotides complementary to EWS-FLI1 mRNA results in decreased proliferation(6-8), suggesting a potential therapeutic intervention directed at this tumor-specific chimeric gene. Small interfering RNAs (siRNAs) have recently been shown to silence the EWS-FLI1 gene in an EFT cell line in vitro(9-11), but the therapeutic efficacy of siRNAs is yet to be demonstrated in vivo.

Systemic applications of virally delivered siRNA and related RNA interference (RNAi) products are unlikely to be viable in the near future because of host immune responses upon repeated delivery and ineffective tumor targeting. The systemic, non-viral delivery of RNAi molecules has been reported in mice and initially involved high-pressure, high-volume tail-vein injections of naked nucleic acid(12-14); a method untenable and unacceptable in humans in routine clinical settings. Subsequently, naked siRNA(15-17), lipid-formulated siRNA(18) and plasmids expressing short hairpin RNA(19, 20) and polycation-formulated siRNA(21-23) have been administered systemically in mice. Naked or formulated siRNAs have also been directly injected into xenograft tumors in mice(24-27). Naked siRNAs require chemical stabilization for in vivo use(17, 28), have non-specific biodistributions that are the same as single-stranded antisense agents(29) and require large and repeated dosages for efficacy(17).

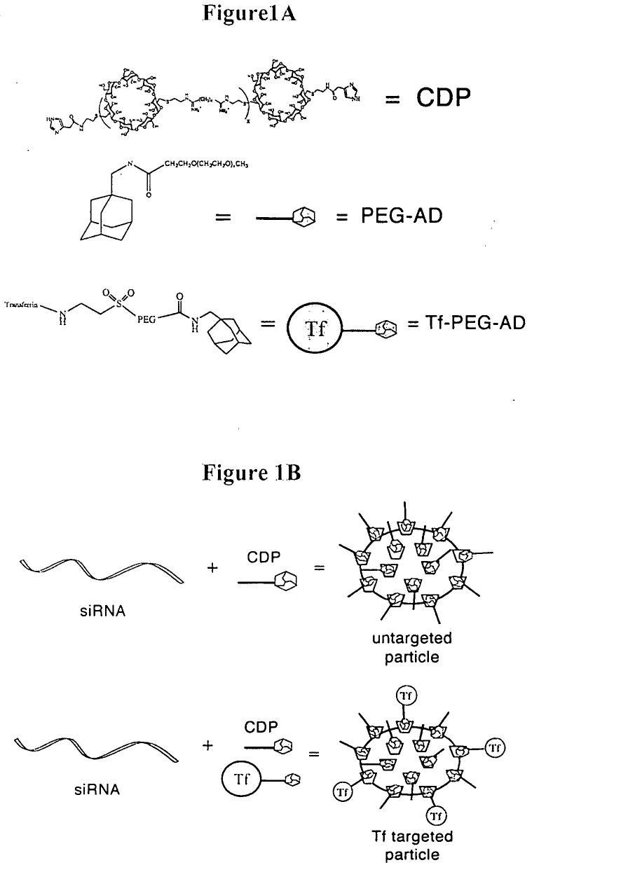

Here, we have used a non-viral delivery system suitable for systemic use, some details of which have been describe (30, 31, 32). The multi-component delivery system includes short polycations containing cyclodextrins that provide low toxicity and enable assembly with the other components of the delivery system that contain targeting ligands (FIG. 1). The cyclodextrin-containing polycations (CDPs) self-assemble with siRNA to form colloidal particles about 50 nm in diameter, and their terminal imidazole groups assist in the intracellular trafficking and release of the nucleic acid(32). CDP protects the siRNA from degradation so that chemical modification of the nucleic acid is unnecessary. The colloidal particles are stabilized for use in biological fluids by surface decoration with polyethylene glycol (PEG) that occurs via inclusion complex formation between the terminal adamantane and the cyclodextrins; some of the PEG chains contain targeting ligands for specific interactions with cell-surface receptors (FIG. 1a). Here, we use transferrin (Tf) as the targeting ligand(33) since tumor cells often overexpress the cell-surface transferrin receptor (TfR)(34). The complete formulation of the siRNA-containing particles is performed by mixing the components together and allowing for the self-assembly as schematically illustrated in FIG. 1b.

By using in vivo, whole-body fluorescence imaging, this system has been shown to deliver fluorescently-labeled single-stranded DNA to tumor cells in subcutaneous, tumor-bearing nude mice from tail-vein injections(35). Absence of the Tf ligand on the particles still provided tumor localization, but no uptake in tumor cells was observed(33, 35).

The safety of siRNA therapy in animals and ultimately humans has also been questioned, especially with regard to triggering interferon-mediated immune responses(36, 37). We recently showed that naked siRNA can be safely administered to mice without eliciting an interferon response(38). Thus far, there are no studies of either the systemic, non-viral delivery of RNAi molecules in a metastatic tumor model or the safety of non-viral, systemic administration of formulated siRNA. Here, we show the safe, systemic, non-viral delivery of RNAi molecules. In particular, systemically delivered siRNA against EWS-FLI1 is shown to inhibit growth and dissemination of EFT cells in vivo.

In order to demonstrate safe, systemic efficacy of non-virally delivered siRNA, we first developed a mouse model of metastatic EFT in NOD/scid mice by tail-vein injections of EFT cells engineered to constitutively express luciferase. The fate of tumor cells was followed by in vivo, whole-body imaging. We tested the ability of targeted, non-viral delivery of siRNA against EWS-FLI1 to safely limit bulk metastatic tumor growth and prevent establishment of bulk metastatic disease from microscopic metastatic disease. We prove here the hypothesis that the targeted, non-viral delivery of siRNA can safely abrogate EWS-FLI1 expression and inhibit metastatic Ewing's tumor growth in vivo.

siRNA Sequences

siRNA targeting luciferase (siGL3), the breakpoint of EWS-FLI1 (siEFBP2), a mutated negative control for siEFBP2 (siEFBP2mut), and a non-targeting control sequence (siCON1) were obtained from Dharmacon Research, Inc. All came purified and pre-annealed (“Option C”). The sequences are: siGL3:

siGL3:
5′-----CUUACGCUGAGUACUUCGAdTdT
dTdTGAAUGCGACUCAUGAAGCU-----5′
siEFBP2(9):
5′---GCAGAACCCUUCUUAUGACUU
UUCGUCUUGGGAAGAAUACUG---5′
siEFBP2mut(9):
5′---GCAGAACCAGUCUUAUGACUU
UUCGUCUUGGUCAGAAUACUG---5′
siCON1:
5′---UAGCGACUAAACACAUCAAUU
UUAUCGCUGAUUUGUGUAGUU---5′

In Vitro Down-Regulation of EWS-FLI1 in an EFT Cell Line

TC71 cells were grown on 6-well plates in RPMI 1640 with 10% FBS (no antibiotics) until they reached 30% confluency. siRNA was complexed with Oligofectamine (OFA, Invitrogen) according to the manufacturer's recommendations or with imidazole-terminated cyclodextrin-containing polycation (CDP) at a 3/1 (+/−) charge ratio(32). The resulting formulations were applied to each well at a final concentration of 100 nM. All transfected cells were harvested at 48 h and gene expression was assessed by Western blot analysis. Primary monoclonal antibodies against the C-terminal region of FLI1 were obtained from BD Biosciences. Polyclonal antibodies against β-Actin were obtained from Santa Cruz Biotechnology.

Determination of Relative Surface Transferrin Receptor (TfR) Level in TC71 Cells

TC71, A2780, and HeLa (the latter two cell lines from American Type Culture Collection) cells were analyzed for relative levels of transferrin receptor (TfR) expression. Cells were plated at 300,000/well in 6-well plates 24 h before exposure to 1 mL of antibiotic-free culture medium containing 1% BSA and various concentrations of fluorescein-labeled transferrin as described previously(35) (50, 100, or 250 nM) for 1 h at 37° C. The cells were washed twice with phosphate-buffered saline (PBS), collected by trypsin treatment, washed twice in FACS buffer (25 mL of Hank's Buffered Salt Solution supplemented with 2 mM MgCl₂ and containing 10 mL DNase) and resuspended in Hank's Buffered Salt Solution for analysis by flow cytometry using a FACSCalibur (Becton Dickinson).

Transduction of TC71 Cells with Luciferase

SMPU-R-MNCU3-LUC is a lentiviral vector based upon HIV-1 that transduces the firefly luciferase gene. The backbone vector SMPU-R has deletions of the enhancers and promoters of the HIV-1 LTR (SIN), has minimal HIV-1 gag sequences, contains the cPPT/CTS sequence from HIV-1, has 3 copies of the UES polyadenylation enhancement element from SV40, and a minimal HIV-1 RRE (gift of Paula Cannon, Children's Hospital Los Angeles) (39). The vector has the U3 region from the MND retroviral vector as an internal promoter driving expression of the firefly luciferase gene from SP-LUC+ (Promega#E178A) (40).

TC71 cells were transduced with viral supernatant containing SMPU-R-MNCU3-LUC vector(41). A second cycle of transduction was performed 8 h later by removing old medium and adding new virus supernatant and medium. Twenty-four hours after the initial transduction, cells were thoroughly washed 3 times with PBS before in vitro analysis.

Injection of Mice with TC71-LUC (Luciferase-Expressing TC71) Cells

TC71-LUC cells were grown in RPMI 1640 with 10% FBS and antibiotics (penicillin/streptomycin). To prepare for injection, cells were trypsinized from the tissue culture flasks and washed twice with PBS. Cells were counted on a hemacytometer slide and resuspended in serum free, antiobiotic-free medium immediately prior to injection. The viability of the cells was tested by trypan blue exclusion. Only cells more than 90% viable were used.

Mice were treated according to the NIH Guidelines for Animal Care and as approved by the Caltech Institutional Animal Care and Use Committee. All mice were 6-8 weeks of age at the time of injection. Each mouse was injected with 5×10⁶ TC71-LUC cells suspended in 0.2 mL RPMI (without FBS or antibiotics) through the tail vein using a 27-gauge needle. All experimental manipulations with the mice were performed under sterile conditions in a laminar flow hood.

Bioluminescent Imaging of the Mice

After the injection of cells, the mice were imaged at different time points using an in vivo IVIS 100 bioluminescence/optical imaging system (Xenogen). D-luciferin (Xenogen) dissolved in PBS was injected intraperitoneally at a dose of 150 mg/kg 10 min before measuring the light emission. General anesthesia was induced with 5% isoflurane and continued during the procedure with 2.5% isoflurane introduced via a nose cone.

After acquiring photographic images of each mouse, luminescent images were acquired with various (1-60 s) exposure times. The resulting grayscale photographic and pseudo-color luminescent images were automatically superimposed by the IVIS Living Image (Xenogen) software to facilitate matching the observed luciferase signal with its location on the mouse. Regions of Interest (ROI) were manually drawn around the bodies of the mice to assess signal intensity emitted. Luminescent signal was expressed as photons per second emitted within the given ROI. Tumor bioluminescence in mice has been shown to be linearly correlated with the tumor volume(42, 43) and we have verified these findings.

Formulation of Non-Viral, siRNA Containing Polyplexes for In Vivo Administration

All complexes were made with siRNA and an imidazole-modified cyclodextrin-containing polycation (CDP), synthesized as described previously(31). Prior to addition to siRNA, CDP was mixed with an adamantane-polyethylene glycol₅₀₀₀ (AD-PEG) conjugate at a 1:1 AD:β-CD (mol:mol) ratio. Targeted polyplexes also contained transferrin-modified. AD-PEG (AD-PEG-Tf) at a 1:1000 AD-PEG-Tf:AD-PEG (w:w) ratio. This mixture was then added to an equal volume of siRNA at a charge ratio (positive charges from CDP to negative charges from siRNA backbone) of 3/1 (+/−). An equal volume of 10% (w/v) glucose in water was added to the resulting polyplexes to give a final polyplex formulation in 5% (w/v) glucose (D5W) suitable for injection.

Consecutive-Day Delivery of siRNA to Tumors In Vivo

Mice with successful tumor cell engraftment received injection of formulations containing siRNA against luciferase (siGL3), EWS-FLI1 (siEFBP2) or a control sequence (siCON1) on two or three consecutive days as indicated. Each mouse (˜20 g) received 0.2 mL of the appropriate formulation, containing 50 μg of siRNA corresponding to a 2.5 mg/kg dose, by low-pressure tail-vein injection using a 1-mL syringe and a 27-gauge needle.

Real Time Quantitative RT-PCR (Q-RT-PCR)

Total cellular RNA was isolated using RNA STAT-60 (Tel-Test) from homogenized tumors. cDNA was synthesized from 2 μg of DNase I (Invitrogen)-treated total RNA in a 42 μl reaction volume using oligo-dT and Superscript II (Invitrogen) for 60 min at 42° C. following suppliers' instructions. PCR primers were designed with MacVector 7.0 (Accelrys). The sequences are:

EWS-FLI1, forward,
5′-CGACTAGTTATGATCAGAGCAGT-3′,
reverse,
5′-CCGTTGCTCTGTATTCTTACTGA-3′;
β-Actin, forward,
5′-GCACCCCGTGCT GCTGAC-3′,
reverse,
5′-CAGTGGTACGGCCAGAGG-3′.

PCR was performed as described before(44). PCR conditions were 95° C. 900 s; 40 cycles of 95° C. 15 s, 60 C 30 s, 72° C. 30 s; and a final denaturing stage from 60° C. to 95° C. All PCR products were analyzed on 1% agarose gel and single band was observed except negative controls. The reproducibility was evaluated by at least three PCR measurements. The expression level of target gene was normalized to internal β-actin and the mean and standard deviation of the target/β-actin ratios were calculated for sample-to-sample comparison.

Long-Term Delivery of siRNA to Tumors In Vivo

Fifty female NOD/scid mice were injected with 5×10⁶ TC71-LUC cells as described above. Immediately after cell injection, each mouse received an additional injection of 0.2 mL of one of the following formulations (concentrations indicated above, 10 mice per group): D5W only (group A); naked siEFBP2 only (group B); targeted, formulated siCON1 (group C); targeted, formulated siEFBP2 (group D); or non-targeted, formulated siEFBP2 (group E). Formulations were administered twice-weekly for four weeks. Images were taken immediately after the first injections for quality control of the injections and twice-weekly immediately before the injection of the formulations. We continued to monitor the tumor signal in the mice receiving targeted (group D) and non-targeted (group E) siEFBP2 formulations for an additional three weeks or until the tumor burden was too great for the mice.

Magnetic Resonance Imaging

Before imaging, each mouse received 100 μL paramagnetic contrast agent MAGNEVIST (1 mL MAGNEVIST contains 469.01 mg gadopentate dimeglumine, 0.99 mg meglumine and 0.4 mg diethylentriamine pentaacetic acid) intraperitoneally to enhance delineation. Mice were sedated with 5% isoflurane and wrapped in cellophane to prevent hypothermia and minimize contamination of the MRI system. Isoflurane gas (0.8% in air) was used for supplementary sedation as needed. All images were obtained using a BRUKER BIOSPIN MRI with a horizontal magnet of 7.0 Tesla (Bruker Instruments, Inc.).

Toxicity, Immune Response, and Pathology Studies

Female C57BL/6 mice (Jackson Laboratories) were 6-8 weeks of age at the time of injection. To measure plasma cytokine levels, blood was harvested from mice 2 h and 24 h post-injection by cardiac puncture and plasma was isolated using Microtainer tubes (Becton Dickinson). Whole blood was used for complete blood count (CBC) analyses, and plasma was used for all liver enzyme and cytokine analyses. IL-12 (p40) (BD Biosciences) and IFN-α levels (PBL Biomedical Laboratories) were measured by ELISA according to the manufacturer's instructions. Major organs of the NOD/scid mice after long-term treatment studies were collected, formalin-fixed and processed for routine hematoxylin and eosin staining using standard methods. Images were collected using a Nikon epifluorescent microscope with a DP11 digital camera.

Results

siRNA Mediates Down-Regulation of EWS-FLI1 in Cultured TC71 EFT Cells

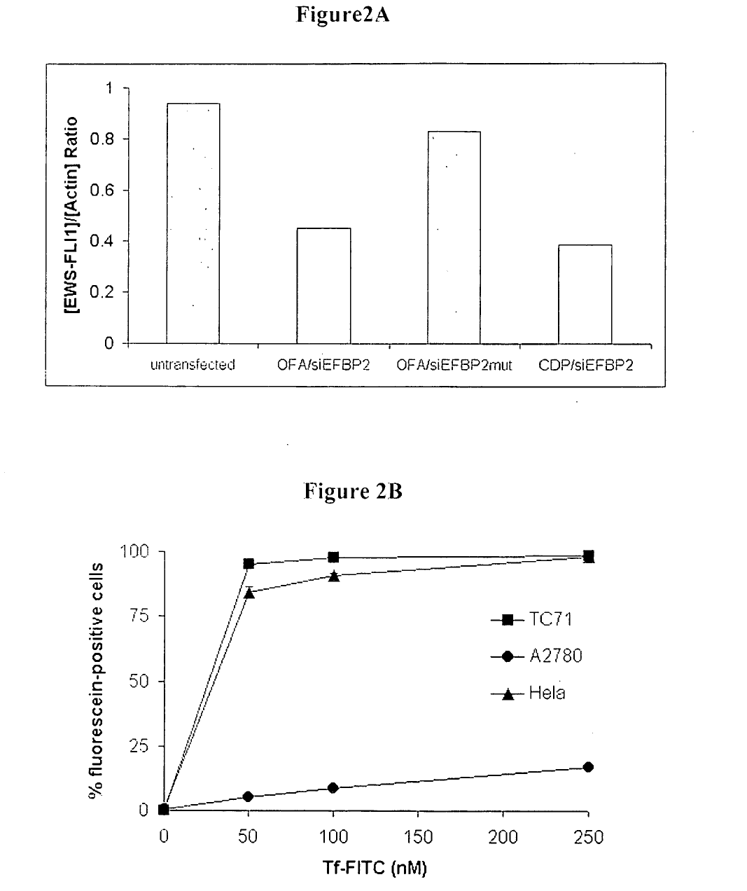

RNAi-mediated gene silencing in TC71, an EFT cell line that expresses the EWS-FLI1 fusion gene, was assessed using a commercial lipid reagent (Oligofectamine, OFA) and our imidazole-terminated cyclodextrin-containing polycation (CDP). Using a previously reported siRNA sequence targeting the EWS-FLI1 breakpoint (siEFBP2)(9), we observed comparable and significant (greater than 50%) reduction in EWS-FLI1 protein levels with both delivery methods (FIG. 2a). Delivery of a mutant siRNA sequence (siEFBP2mut) failed to elicit such down-regulation.

TC71 Cells Display a High Relative Surface Transferrin Receptor (TfR) Level

The level of the cell-surface transferrin receptor (TfR) in TC71 cells was determined relative to cell lines previously shown to have high (HeLa) and low (A2780) TfR levels(35) (FIG. 2b). By 50 nM concentration, we observed 100% uptake of a FITC-transferrin (FITC-Tf) conjugate by TC71 cells, even higher than that by HeLa cells at all FITC-Tf concentrations examined. These results suggest that modification of siRNA formulations to contain a ligand for TfR could lead to successful targeting to TC71 cells in vivo.

Establishment of a Murine Model of Metastatic Ewing's Sarcoma

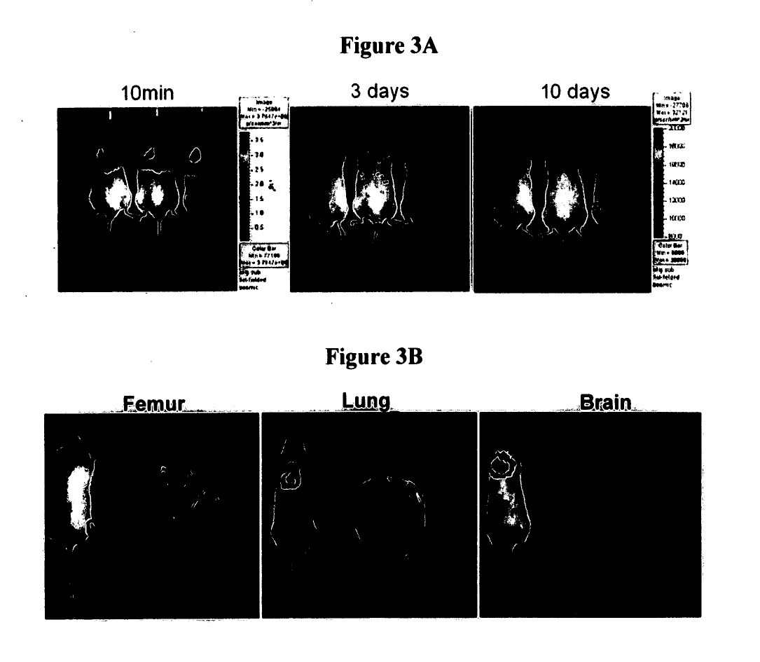

Luciferase-expressing TC71 cells (TC71-LUC) were generated by viral transduction and administered to female NOD/scid mice by tail-vein injection. The pattern of TC71-LUC cell engraftment was assessed by acquiring serial images of in vivo bioluminescence for 5-8 weeks after transplantation. Signals could be detected immediately after the transplantation. Ten minutes after cell injection, the luminescence signals accumulated in the lung area, indicative of entrapment of TC71-LUC cells within the capillary bed of the lung (FIG. 3a). Over the next few hours, the bioluminescent signal gradually disappeared as the cells dispersed and reemerged one to two weeks later at various locations where tumors developed. The most common engraftment sites were lung, vertebral column, pelvis, femur and soft tissue, similar to the most frequently observed sites for metasases in EFT patients(45). The locations of the engraftments were confirmed by MRI (FIG. 3b), CT, X-ray scans, and necropsy with histopathologic confirmation (data not shown).

Formulated siRNA Against Luciferase Transiently Reduces the Bioluminescent Signal of Engrafted Tumors In Vivo

To test whether targeted, systemic CDP-mediated delivery of siRNA could provide gene silencing in vivo, two consecutive daily treatments (days 40 and 41 after cell injection) were performed on mice bearing luciferase-producing metastasized EFT. The tumors of mice treated with the targeted, formulated siGL3-containing polyplexes showed a strong decrease (greater than 90%) in luciferase signal 2-3 days after injection. The luciferase down-regulation was transient. The luminescent signal increased daily thereafter. Heidel et al. have shown that low volume tail vein injections of naked siRNA at 2.5 mg/kg do not give luciferase downregulation in mice most likely due to the lack of cellular uptake of naked siRNAs administered at that dose (32). Taken together, these studies demonstrate that the Tf-targeted, CDP-containing particles can deliver functional siRNA to TC71-LUC tumors when administered via standard low-pressure tail-vein injection.

Formulated siRNA Against EWS-FLI1 Inhibits Tumor Growth In Vivo

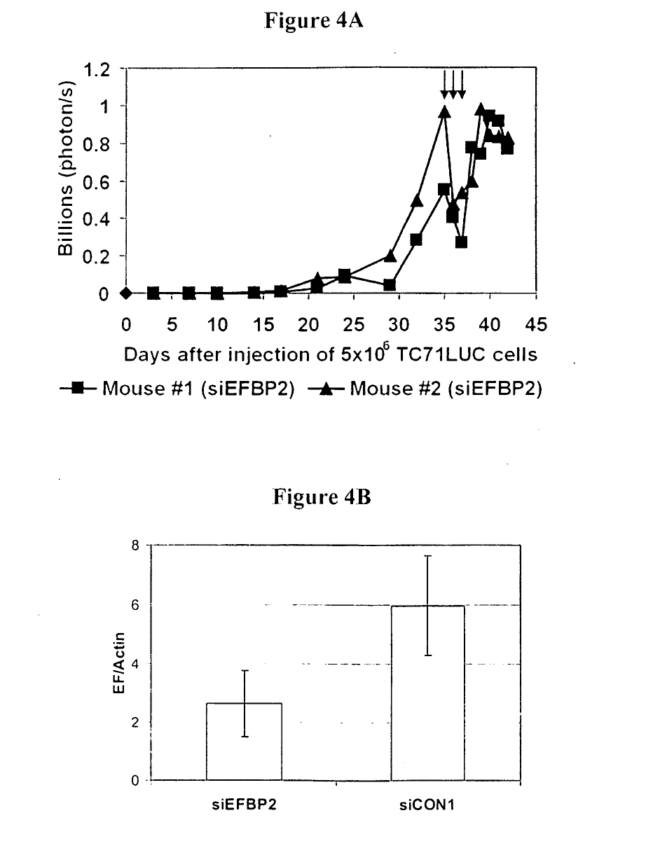

Mice with successful engraftment of TC71-LUC cells were randomly selected for treatment with targeted, formulated siEFBP2 on days 35, 36, and 37 after cell injection. Increases in bioluminescent signal from metastasized tumor growth were inhibited by systemic administration of targeted formulations containing siRNA against EWS-FLI1 (siEFBP2) (FIG. 4a). Three consecutive daily injections of the targeted, formulated siEFBP2 resulted in a decreased tumor signal, and this effect lasted 2-3 days. Further assessment of the EWS-FLI1 expression in the tumors treated with two consecutive siEFBP2 formulations showed a 60% down-regulation of EWS-FLI1 RNA level compared to siCON1-treated tumors (p=0.046). (FIG. 4b). Therefore, the delivery of fully formulated siEFBP2 is able to reduce EWS-FLI1 expression in the established tumors and provide transient inhibition of EFT tumor growth.

Long-Term, Twice-Weekly Administration of Targeted, Formulated siEFBP2 Inhibits Tumor Cell Engraftment

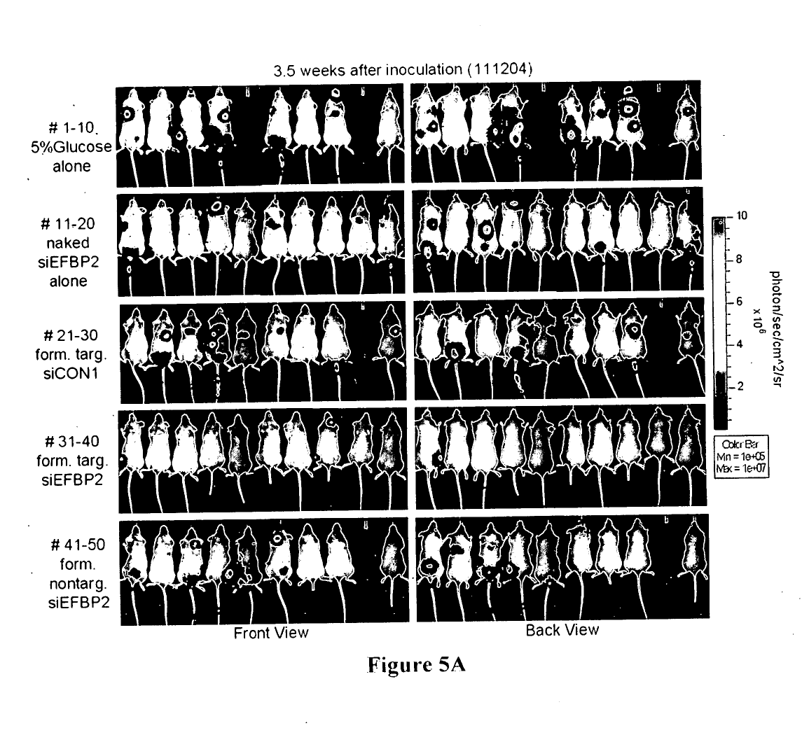

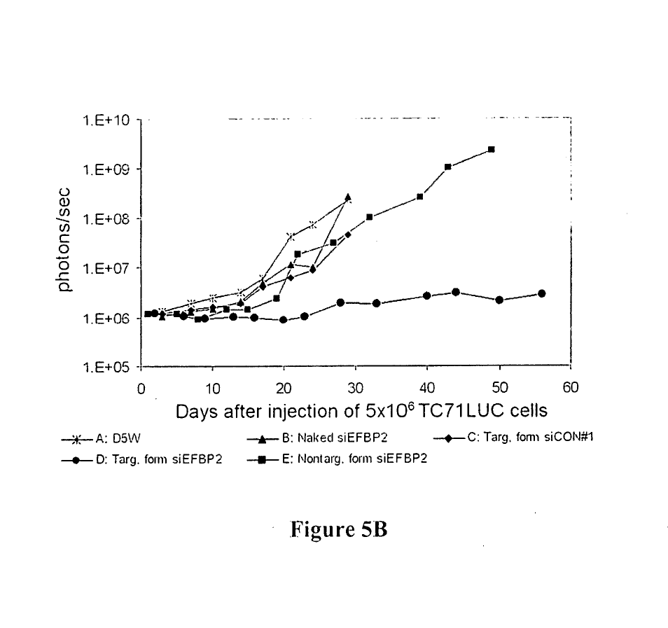

After observing transient effects in vivo after short-term (1-3 daily treatments) administration of targeted siRNA formulations, we employed a long-term treatment regimen in which formulations were administered twice weekly beginning the same day as injection of TC71-LUC cells. These studies allowed for the more careful investigation of the effects of the formulations that included all of the proper controls. The success of tumor cell injection was confirmed by imaging mice immediately after the injection. Targeted, formulated siEFBP2 treatments (group D) dramatically inhibited the engraftment of TC71-LUC cells (FIGS. 5a and 5b), with only 20% of the mice showing any tumor growth compared to 90-100% in other treatment groups (FIG. 5a). Neither the mice receiving naked siEFBP2 (group B) nor those receiving targeted delivery of siCON1 (group C) showed any difference in tumor engraftment compared to the control group that received only the 5% glucose carrier solution (D5W, group A). Interestingly, tumors in mice treated with formulated but non-targeted (lacking Tf) siEFBP2 showed a delayed progression of tumor engraftment compared to the control groups. Once significant tumors were established, however, the tumors seemed to grow at a rate unaffected by continued treatment with the non-targeted siEFBP2 (FIG. 5b). The tumor signal was monitored in the mice receiving targeted (group D) and non-targeted (group E) siEFBP2 formulations for an additional three weeks or until the tumor burden was too great for the mice. Whereas most of the mice receiving non-targeted formulations developed very large tumors, the majority of the mice receiving targeted formulations showed little or no tumor signal (FIG. 5b). We conclude that treatment with the targeted formulation of siEFBP2 prevented the tumor cell engraftment in these mice and slowed the growth of any tumors that did develop. Also, targeted, formulated siEFBP2 complexes do not appear to cross the blood-brain barrier since the tumor growth of a brain metastasis treated by this complex was unaffected. This result is consistent with previously reported biodistribution studies(35).

No Immune Response or Major Organ Damage was Observed after Targeted Formulated siEFBP2 Treatment in Mice

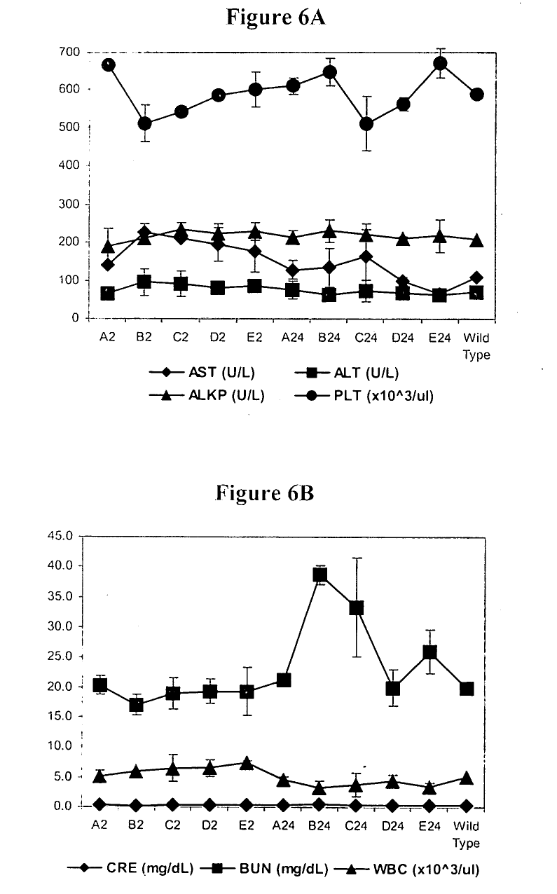

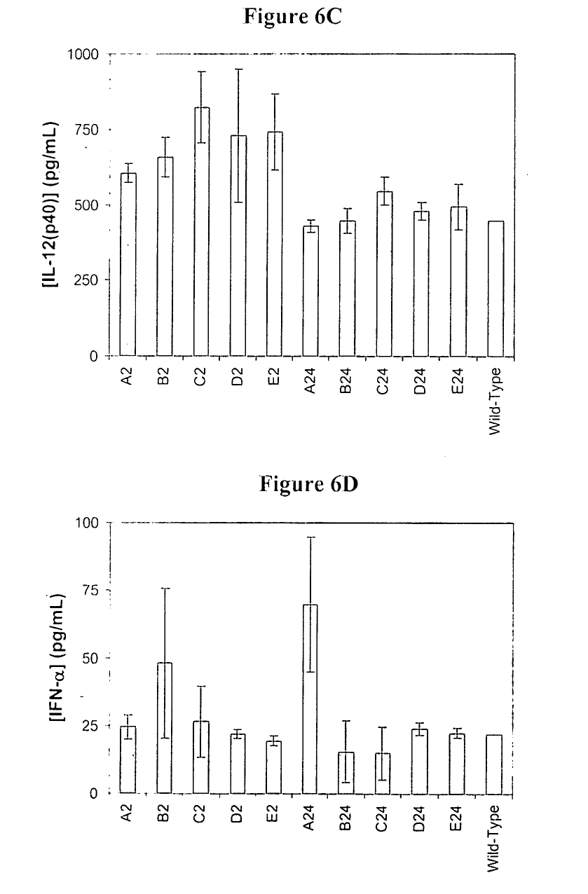

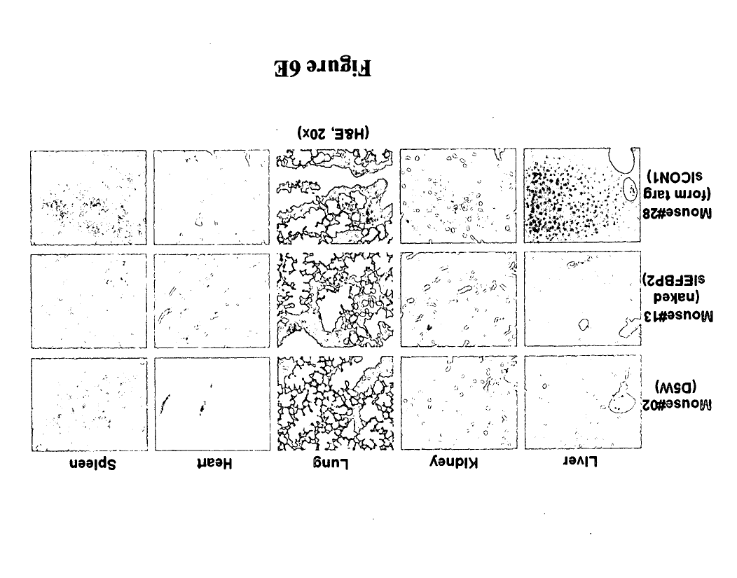

Since the ability of the NOD/scid mice to mount a possible immune response to these formulations is severely compromised, single tail-vein injections of formulations were repeated in immunocompetent mice (C57BL/6) and blood was collected at 2 h or 24 h after the injections. Complete blood counts (CBC) of whole blood showed insignificant changes in white blood cell (WBC) or platelet (PLT) counts (FIG. 6a). Levels of secreted liver enzymes (AST, ALT), blood urea nitrogen (BUN), and creatinine (CRE) were all unchanged, indicating a lack of damage to the liver or kidneys. No increases, resulting from formulations, in plasma interleukin-12 (IL-12) or interferon-alpha (IFN-α) at either 2 h or 24 h post-injection were observed (FIG. 6b). We also performed pathological examination of the major organs (liver, kidney, brain, heart, lung, and pancreas) from the NOD/scid mice that received long-term treatments by hematoxylin and eosin (H&E) staining (FIG. 6c). No organ damage was observed with any of the formulated groups when compared to the D5W and naked siEFBP2 treatment groups. Taken together, these results demonstrate the safety and low immunogenicity of these CDP-containing formulations.

The silencing of gene expression by siRNA is a powerful tool for the genetic analysis of mammalian cells and has the potential for development into specific, potent and safe treatments for human disease. However, delivery of siRNA into specific organs in vivo is a major obstacle for RNAi-based therapy. To overcome this problem, a hydrodynamic method (high-pressure, high-volume tail-vein injection) has been used in mice to deliver siRNA (and other types of nucleotides) to the liver. This method is ineffective for other organs and is not feasible for routine clinical application(14, 46). Naked siRNA has been employed in mice but requires costly chemical stabilization and large, frequent dosing for efficacy(17). While researchers have also shown successful viral delivery of plasmids to achieve prolonged and stable expression of siRNA(47-51), the immunogenicity of viral vectors provide significant barriers to their clinical use. Also, it is difficult to influence the biodistribution of viral vectors and preferentially target tumor when administered systemically. Therefore, the development of a targeted, non-immunogenic siRNA delivery system for systemic administration is highly desired and will likely be required for effective use of siRNA as a human therapy. Here, we show that a cyclodextrin-based polycation delivery system (FIG. 1a) can be formulated (FIG. 1b) to target metastatic cancer in a murine model of the Ewing's family of tumors.

We established a highly reproducible and clinically relevant metastatic murine model for the Ewing's family of tumors in NOD/scid mice (FIG. 3). EFT cells were transduced with the firefly luciferase gene prior to administration in mice, thus allowing for non-invasive, in vivo, whole-body imaging of bioluminescence to monitor the fate of tumor cells. The tumor engraftment sites observed (lung, vertebral column, pelvis, femur and soft tissue) were comparable to the most common locations of metastases in EFT patients.

Small interfering RNA (siRNA) duplexes targeting the EWS-FLI1 fusion gene (siEFBP2) or the firefly luciferase gene (siGL3) were formulated with the synthetic delivery system as schematically illustrated in FIG. 1. Since the TC71 cells used here were shown to express high levels of cell-surface transferrin receptors (FIG. 2b), targeted formulations contained transferrin (Tf) as the targeting ligand. This delivery system self-assembles with siRNA to give ˜50 nm particles that are stable in physiologic fluid, can protect the nucleic acid from nuclease degradation (protection for at least 72 h—data not shown), are capable of providing for cellular uptake and delivery of functional siRNA (FIG. 2a) and can target TfR-expressing tumor cells from tail-vein administration in mice³²⁻³⁶. When introduced systemically into tumor-bearing mice by tail-vein injection, these formulations containing either siEFBP2 or siGL3 are able to achieve transient reduction in tumor growth or luciferase expression, respectively (FIG. 4). The tumor growth inhibition was correlated with a sequence-specific down-regulation of EWS-FLI1 expression in the tumors.

Clinically, many tumors relapse after intensive treatment because of systemic dissemination of micrometastases. Nearly all EFT patients already have micrometastases at diagnosis, resulting in a >95% relapse rate when treated locally (1), and a 40% relapse rate after systemic chemotherapy(2). Therefore, effective treatment for elimination of circulating or dormant metastasized tumor cells after traditional therapy is needed. We explored the possibility of using targeted, formulated siRNA for this purpose by administration of formulations twice-weekly beginning the same day as injection of TC71-LUC cells. These injections of the different formulations in tumor-bearing NOD/scid mice reveal that only the targeted, formulated siEFBP2 achieves long-term tumor growth inhibition (FIG. 5). Neither naked siEFBP2 nor a formulated control siRNA sequence shows any effect on tumor signal compared to the control group receiving only the carrier fluid. These results demonstrate the necessity of the delivery vehicle for systemic application and the sequence-specificity of the observed inhibition

Notably, mice treated with formulated but non-targeted siEFBP2 show an initial delay in tumor growth. However, the growth rate of tumors that eventually developed are unaffected by continuation of this treatment The enhanced permeability and retention effect (EPR) leads to the accumulation of macromolecules in solid tumors(52). The leaky vasculature associated with the nascent tumors allows circulating targeted and non-targeted particles to accumulate in tumors. However, only the Tf-containing, targeted particles were detected within tumor cells by fluorescence(35). Some small fraction of the non-targeted particles may have entered tumor cells. If so, their amount was below the detection limit. Mice receiving non-targeted formulations in the present study eventually develop very large tumors while little or no tumor signal is observed by imaging or at autopsy in most mice receiving the targeted formulations. These results show that Tf targeting increases overall uptake of the nanoparticles through receptor-specific endocytosis by tumor cells after accumulation in the tumor mass via the EPR effect has occurred.

Treatment with the targeted formulation of siEFBP2 assists in the prevention of the initial establishment of tumors in these mice from the injected cells and slows the growth of any tumors that develop by down-regulating the expression of the oncogenic fusion protein EWS-FLI1. Because the siGL3-containing formulations show potent, sequence-specific down-regulation of in vivo bioluminescence, it is clear that the delivered siRNA is functional. While the luciferase down-regulation is a direct observation of in vivo RNAi, the reduced tumor engraftments from siEFBP2-containing formulations require a more extended cascade of down-regulation and intracellular signal transduction events and are therefore indirect, but biologically significant, measures of sequence-specific RNAi.

Most of the tumor engraftment sites in the mouse model match those commonly seen in EFT-bearing patients. We also observed brain metastases, analogous to that rare event in human EFT patients (FIGS. 3b and 5a). As expected, previous work with this delivery system showed that these formulations are unable to cross the blood-brain barrier(35) and as such we would not expect them to reduce growth of brain metastases. Indeed, the targeted, formulated siEFBP2 complexes did not appear to affect the tumor growth of the illustrated brain metastasis.

Recent in vitro reports have shown that siRNA sequences and their method of delivery may trigger an interferon response(36, 37). Additionally, in vivo delivery of siRNA by lipids have resulted in potent interferon responses (53-55). Here, single tail-vein injections of all of the formulations were performed in immunocompetent (C57BL/6) mice to enable measurement of numerous blood markers that are indicative of an immune response. In contrast to results obtained from the injection of poly (I:C), a known immunostimulator through interactions with Toll-like receptor 3 (TLR3)(38), none of the formulations show any significant effects on the levels of IL-12, IFN-α, white blood cells, platelets, secreted liver enzymes (ALT and AST), BUN, or CRE (FIG. 6). All of these observations with formulated siRNA are consistent with our previous work showing a lack of immune response to naked siRNA(38). The cyclodextrin-based delivery system does not produce an interferon response even when siRNA is used that contains a motif known to be immunostimulatory when delivered in vivo with lipids (54) (published sequence is within siCON1). These results show the safety and low immunogenicity of CDP-containing formulations and demonstrate the attractiveness of this methodology for systemic, targeted delivery of nucleic acids. The in vivo gene silencing effect of siRNA by our delivery system is transient, permitting fine-tuning of the intensity and interval of the treatment. For example, the frequency of administration can be tuned for use in combination with other agents, and the treatment can be terminated within a few days if necessary.

We have demonstrated that systemic administration of siRNA can provide safe, sequence-specific inhibition of tumor growth in a disseminated tumor model. In contrast to naked siRNA delivery, the targeted siRNA formulations used here are efficacious at low siRNA doses and do not require chemical modification of the siRNA for stabilization. Further, this delivery system can be easily tuned to target different cell-surface receptors in tumors and other tissue(32), can be used to deliver different and/or multiple siRNA sequences, and does not elicit a detectable immune response or any changes in mouse physiology. We believe this treatment has the potential to be developed into a useful method for inhibition of metastatic EFT growth and may also have broad applicability in cancer therapy. Future experiments using an EFT-specific targeting ligand and employing formulation combinations with small-molecule drugs will likely further enhance the anti-tumoral potency of this system.

Although the invention has been described with reference to embodiments and examples, it should be understood that various modifications can be made without departing from the spirit of the invention. Accordingly, the invention is limited only by the following claims. All references cited herein are hereby expressly incorporated by reference.

### REFERENCES

1. Dahlin D C, Coventry M B, Scanlon P W Ewing's sarcoma. A critical analysis of 165 cases. Am J Orthop 1961; 43-A:185-192.
2. Rodriguez-Galindo C, Spunt S L, Pappo A S Treatment of Ewing sarcoma family of tumors: current status and outlook for the future. Med Pediatr Oncol 2003; 40:276-287.
3. Delattre O, Zucman J, Melot T, et al. The Ewing family of tumors—a subgroup of small-round-cell tumors defined by specific chimeric transcripts. N Engl J Med 1994; 331:294-299.
4. Bailly R, Bosselut R, Zucman J, et al. DNA-binding and transcriptional activation properties of the EWS-FLI-1 fusion protein resulting from the t(11;22) translocation in Ewing sarcoma. Mol. Cell. Biol. 1994; 14:3230-3241.
5. Ohno T, Rao V, Reddy E EWS/FLI-1 chimeric protein is a transcriptional activator. Cancer Res 1993; 53:5859-5863.
6. Tanaka K, Iwakuma T, Harimaya K, et al. EWS-FLI1 antisense oligodeoxynucleotide inhibits proliferation of human Ewing's sarcoma and primitive neuroectodermal tumor cells. J Clin Invest 1997; 99:239-247.
7. Maksimenko A, Lambert G, Bertrand J R, et al. Therapeutic potentialities of EWS-FLI-1 mRNA-targeted vectorized antisense oligonucleotides. Ann N Y Acad Sci 2003; 1002:72-77.
8. Maksimenko A, Malvy C, Lambert G, et al. Oligonucleotides targeted against a junction oncogene are made efficient by nanotechnologies. Pharm Res 2003; 20:1565-1567.
9. Dohjima T, Lee N S, Li H, et al. Small interfering RNAs expressed from a Pol III promoter suppress the EWS/FLI-1 transcript in an Ewing sarcoma cell line. Mol Ther 2003; 7:811-816.
10. Kovar H, Ban J, Pospisilova S Potentials for RNAi in sarcoma research and therapy: Ewing's sarcoma as a model. Semin Cancer Biol 2003; 13:275-281.
11. Chansky H A, Barahmand-Pour F, Mei Q, et al. Targeting of EWS/FLI-1 by RNA interference attenuates the tumor phenotype of Ewing's sarcoma cells in vitro. J Orthop Res 2004; 22:910-917.
12. Lewis D L, Hagstrom J E, Loomis A G, et al. Efficient delivery of siRNA for inhibition of gene expression in postnatal mice. Nat Genet 2002; 32:107-108.
13. McCaffrey A P, Meuse L, Pham T T, et al. RNA interference in adult mice. Nature 2002; 418:38-39.
14. Song E, Lee S K, Wang J, et al. RNA interference targeting Fas protects mice from fulminant hepatitis. Nat Med 2003; 9:347-351.
15. Filleur S, Courtin A, Ait-Si-Ali S, et al. siRNA-mediated inhibition of vascular endothelial growth factor severely limits tumor resistance to antiangiogenic thrombospondin-1 and slows tumor vascularization and growth. Cancer Res 2003; 63:3919-3922.
16. Duxbury M S, Matros E, Ito H, et al. Systemic siRNA-mediated gene silencing: a new approach to targeted therapy of cancer. Ann Surg 2004; 240:667-674; discussion 675-666.
17. Soutschek J, Akinc A, Bramlage B, et al. Therapeutic silencing of an endogenous gene by systemic administration of modified siRNAs. Nature 2004; 432:173-178.
18. Sioud M, Sorensen D R Cationic liposome-mediated delivery of siRNAs in adult mice. Biochem Biophys Res Commun 2003; 312:1220-1225.
19. Zhang Y, Boado R J, Pardridge W M In vivo knockdown of gene expression in brain cancer with intravenous RNAi in adult rats. J Gene Med 2003; 5:1039-1045.
20. Zhang Y, Zhang Y F, Bryant J, et al. Intravenous RNA interference gene therapy targeting the human epidermal growth factor receptor prolongs survival in intracranial brain cancer. Clin Cancer Res 2004; 10:3667-3677.
21. Schiffelers R M, Ansari A, Xu J, et al. Cancer siRNA therapy by tumor selective delivery with ligand-targeted sterically stabilized nanoparticle. Nucleic Acids Res 2004; 32:e149.
22. Ge Q, Filip L, Bai A, et al. Inhibition of influenza virus production in virus-infected mice by RNA interference. Proc Natl Acad Sci USA 2004; 101:8676-8681.
23. Urban-Klein B, Werth S, Abuharbeid S, et al. RNAi-mediated gene-targeting through systemic application of polyethylenimine (PEI)-complexed siRNA in vivo. Gene Ther 2005; 12:461-466.
24. Bertrand J R, Pottier M, Vekris A, et al. Comparison of antisense oligonucleotides and siRNAs in cell culture and in vivo. Biochem Biophys Res Commun 2002; 296:1000-1004.
25. Minakuchi Y, Takeshita F, Kosaka N, et al. Atelocollagen-mediated synthetic small interfering RNA delivery for effective gene silencing in vitro and in vivo. Nucleic Acids Res 2004; 32:e109.
26. Takei Y, Kadomatsu K, Yuzawa Y, et al. A small interfering RNA targeting vascular endothelial growth factor as cancer therapeutics. Cancer Res 2004; 64:3365-3370.
27. Pille J Y, Denoyelle C, Varet J, et al. Anti-RhoA and anti-RhoC siRNAs inhibit the proliferation and invasiveness of MDA-MB-231 breast cancer cells in vitro and in vivo. Mol Ther 2005; 11:267-274.
28. Layzer J M, McCaffrey A P, Tanner A K, et al. In vivo activity of nuclease-resistant siRNAs. RNA 2004; 10:766-771.
29. Braasch D A, Paroo Z, Constantinescu A, et al. Biodistribution of phosphodiester and phosphorothioate siRNA. Bioorg Med Chem Lett 2004; 14:1139-1143.
30. Gonzalez H, Hwang S J, Davis M E New class of polymers for the delivery of macromolecular therapeutics. Bioconjug Chem 1999; 10:1068-1074.
31. Pun S H, Davis M E Development of a nonviral gene delivery vehicle for systemic application. Bioconjug Chem 2002; 13:630-639.
32. Davis M E, Pun S H, Bellocq N C, et al. Self-assembling nucleic acid delivery vehicles via linear, water-soluble, cyclodextrin-containing polymers. Curr Med Chem 2004; 11:179-197.
33. Bellocq N C, Pun S H, Jensen G S, et al. Transferrin-containing, cyclodextrin polymer-based particles for tumor-targeted gene delivery. Bioconjug Chem 2003; 14:1122-1132.
34. Gatter K C, Brown G, Trowbridge I S, et al. Transferrin receptors in human tissues: their distribution and possible clinical relevance. J Clin Pathol 1983; 36:539-545.
35. Pun S H, Tack F, Bellocq N C, et al. Targeted Delivery of RNA-Cleaving DNA Enzyme (DNAzyme) to Tumor Tissue by Transferrin-Modified, Cyclodextrin-Based Particles. Cancer Biol Ther 2004; 3:641-650.
36. Sledz C A, Holko M, de Veer M J, et al. Activation of the interferon system by short-interfering RNAs. Nat Cell Biol 2003; 5:834-839.
37. Bridge A J, Pebernard S, Ducraux A, et al. Induction of an interferon response by RNAi vectors in mammalian cells. Nat Genet 2003; 34:263-264.
38. Heidel J D, Hu S, Liu X F, et al. Lack of interferon response in animals to naked siRNAs. Nat Biotechnol 2004; 22:1579-1582.
39. Bahner I, Kearns K, Hao Q L, et al. Transduction of human CD34+ hematopoietic progenitor cells by a retroviral vector expressing an RRE decoy inhibits human immunodeficiency virus type 1 replication in myelomonocytic cells produced in long-term culture. J Virol 1996; 70:4352-4360.
40. Challita P M, Skelton D, el-Khoueiry A, et al. Multiple modifications in cis elements of the long terminal repeat of retroviral vectors lead to increased expression and decreased DNA methylation in embryonic carcinoma cells. J Virol 1995; 69:748-755.
41. Wang X, Rosol M, Ge S, et al. Dynamic tracking of human hematopoietic stem cell engraftment using in vivo bioluminescence imaging. Blood 2003; 102:3478-3482.
42. Rehemtulla A, Stegman L D, Cardozo S J, et al. Rapid and quantitative assessment of cancer treatment response using in vivo bioluminescence imaging. Neoplasia 2000; 2:491-495.
43. Vooijs M, Jonkers J, Lyons S, et al. Noninvasive imaging of spontaneous retinoblastoma pathway-dependent tumors in mice. Cancer Res 2002; 62:1862-1867.
44. Zhang J, Hu S, Schofield D E, et al. Selective usage of D-Type cyclins by Ewing's tumors and rhabdomyosarcomas. Cancer Res 2004; 64:6026-6034.
45. Vormoor J, Baersch G, Decker S, et al. Establishment of an in vivo model for pediatric Ewing tumors by transplantation into NOD/scid mice. Pediatr Res 2001; 49:332-341.
46. Sorensen D R, Leirdal M, Sioud M Gene silencing by systemic delivery of synthetic siRNAs in adult mice. J Mol Biol 2003; 327:761-766.
47. Hasuwa H, Kaseda K, Einarsdottir T, et al. Small interfering RNA and gene silencing in transgenic mice and rats. FEBS Lett 2002; 532:227-230.
48. Miyagishi M, Taira K Development and application of siRNA expression vector. Nucleic Acids Res Suppl 2002113-114.
49. Pekarik V, Bouri D, Miglino N, et al. Screening for gene function in chicken embryo using RNAi and electroporation. Nat Biotechnol 2003; 21:93-96.
50. Hemann M T, Fridman J S, Zilfou J T, et al. An epi-allelic series of p53 hypomorphs created by stable RNAi produces distinct tumor phenotypes in vivo. Nat Genet 2003; 33:396-400.
51. Rubinson D A, Dillon C P, Kwiatkowski A V, et al. A lentivirus-based system to functionally silence genes primary mammalian cells, stem cells and transgenic mice by RNA interference. Nat Genet 2003; 33:401-406.
52. Tanaka T, Shiramoto S, Miyashita M, et al. Tumor targeting based on the effect of enhanced permeability and retention (EPR) and the mechanism of receptor-mediated endocytosis (RE). Int J Pharm 2004; 277:39-61.
53. Homung V, Guenthner-Biller M, Bourquin C, et al. Sequence-specific potent induction of IFN-alpha by short interfering RNA in plasmacytoid dendritic cells through TLR7. Nat Med 2005; 11:263-270.
54. Judge A D, Sood V, Shaw J R, et al. Sequence-dependent stimulation of the mammalian innate immune response by synthetic siRNA. Nat Biotechnol 2005; 23:457-462.
55. Ma Z, Li J, He F, et al. Cationic lipids enhance siRNA-mediated interferon response in mice. Biochem Biophys Res Commun 2005; 330:755-759.

1. (canceled)

2. A composition for delivering a therapeutic agent comprising colloidal particles that include an imidazole-terminated cyclodextrin polycation, an adamantane-terminated polyethylene glycol, and a therapeutic agent for selective inhibition of Ewing's Family Tumor (EFT) growth.

3. The composition of claim 2 wherein the colloidal particles have an average diameter of less than about 100 nm.

4. The composition of claim 3 wherein the colloidal particles have an average diameter of greater than about 10 nm.

5. The composition of claim 2 wherein the therapeutic agent for selective inhibition of Ewing's Family Tumor (EFT) growth is an antisense oligonucleotide that selectively binds to the EWS-FLI1 gene.

6. A composition for delivering a therapeutic agent for selective inhibition of Ewing's Family Tumor (EFT) growth comprising colloidal particles that include an imidazole-terminated cyclodextrin polycation, an adamantane-terminated polyethylene glycol, and a small interfering RNA (siRNA) that selectively binds to mRNA of an oncogene associated with Ewing's Family Tumors.

7. The composition of claim 6 wherein the colloidal particles have an average diameter of less than about 100 nm.

8. The composition of claim 7 wherein the colloidal particles have an average diameter of greater than about 10 nm.

9. The composition of claim 6 wherein the oncogene is the EWS-FLI1 gene.

10. The composition of claim 9 wherein the siRNA is a double stranded RNA segment having the following structure:
5′-GCAGAACCCUUCUUAUGACUUUUCGUCUUGGGAAGAAUACUG-5′.

11. The composition of claim 9 wherein the siRNA is a double stranded RNA segment having the following structure:
5′-GCAGAACCAGUCUUAUGACUUUUCGUCUUGGUCAGAAUACUG-5′.

12. The composition of claim 9 wherein the adamantane-terminated polyethylene glycol includes a targeting group bound thereto that selectively binds to a cell surface antigen expressed on Ewing's Family Tumors.

13. The composition of claim 12 wherein the cell surface antigen is transferrin receptor and the targeting group is transferrin.

14. A method for treating a patient suffering from Ewing's Family Tumors (EFT) comprising administering to a patient suffering from EFT a therapeutically effective amount of a composition comprising colloidal particles that include an imidazole-terminated cyclodextrin polycation, an adamantane-terminated polyethylene glycol, and a small interfering RNA (siRNA) that selectively binds to mRNA of an oncogene associated with Ewing's Family Tumors.

15. The method of claim 14 wherein the colloidal particles have an average diameter of less than about 100 nm.

16. The method of claim 15 wherein the colloidal particles have an average diameter of greater than about 10 nm.

17. The method of claim 14 wherein the oncogene is the EWS-FLI1 gene.

18. The method of claim 17 wherein the siRNA is a double stranded RNA segment having the following structure:
5′-GCAGAACCCUUCUUAUGACUUUUCGUCUUGGGAAGAAUACUG-5′.

19. The method of claim 17 wherein the siRNA is a double stranded RNA segment having the following structure:
5′-GCAGAACCAGUCUUAUGACUUUUCGUCUUGGUCAGAAUACUG-5′.

20. The method of claim 14 wherein the adamantane-terminated polyethylene glycol includes a targeting group bound thereto that selectively binds to a cell surface antigen expressed on Ewing's Family Tumors.

21. The method of claim 20 wherein the cell surface antigen is transferrin receptor and the targeting group is transferrin.
